(19) United States
(12) Patent Application Publication
(10) Pub. No.: US 2017/0188930 A1
(43) Pub. Date: Jul. 6, 2017

(54) ANIMATION-BASED AUTISM SPECTRUM DISORDER ASSESSMENT

(71) Applicant: Oregon Health & Science University, Portland, OR (US)

(72) Inventor: Garet Lahvis, Portland, OR (US)

(21) Appl. No.: 15/510,657

(22) PCT Filed: Sep. 10, 2015
(86) PCT No.: PCT/US2015/049479

§ 371 Date: Mar. 10, 2017

Related U.S. Application Data
(60) Provisional application No. 62/048,611, filed on Sep. 10, 2014.

(51) Int. Cl. A61B 5/16; A61B 5/11; A61B 5/00; A61B 3/113

(52) U.S. Cl. CPC A61B 5/168; A61B 3/113; A61B 5/1118; A61B 5/4833

(57) ABSTRACT

Systems and methods for quantitative assessment of autism spectrum disorder based on detected responses of a subject to displayed animations are disclosed. In one example approach, a method for performing an autism assessment of a subject includes presenting as animation on a display, tracking the subject's eye gaze location on the display during specific scenes occurring within the animation where the specific scenes are associated with an autism construct, calculating an amount of time the subject's eye gaze tracks to predetermined regions on the display within the specific scenes to obtain a calculated eye gaze time associated with the specific scenes and the autism construct, and outputting an indication of the calculated eye gaze time. Animations may be selected to target a plurality of autism constructs and detected responses may be used to generate response mappings across the plurality of autism constructs.

### CROSS REFERENCE TO RELATED APPLICATIONS

The present application claims priority to U.S. Provisional Patent Application No. 62/048,611, filed Sep. 10, 2014, entitled “Animation-Based Autism Spectrum Disorder Assessment,” the entire disclosure of which is hereby incorporated by reference in its entirety.

### ACKNOWLEDGMENT OF GOVERNMENT SUPPORT

This invention was made with United States government support under the terms of grant number UL1TR000128, awarded by the National Institutes of Health. The United States government has certain rights in this invention.

### FIELD

The present disclosure relates to the field of autism spectrum disorder assessment, and, more specifically, to systems and methods for quantitative assessment of an autism spectrum disorder based on detected responses of a subject.

### BACKGROUND

Autism spectrum disorders (ASD) are characterized by abnormalities in reciprocal social interaction and communication and by the presence of circumscribed interests and repetitive behaviors. Examples of typical symptoms of autism include impaired communication due to reduced ability to make eye contact, reduced emotional interaction due to a reduced ability to imagine the feelings of others, perseveration on narrow areas of interest (e.g., president's names and birthdays, the location of stop signs, etc.), and/or repetitive behaviors.

Early identification of ASD can improve a child's long-term health outcome as well as the ability to cope with the disease by those who work with the child, e.g., family, guardians, teachers, etc. Early interventions that involve active and prolonged social engagement have been shown to improve outcomes for children with autism. After initial screening by pediatricians, clinicians with specialized training conduct direct assessments of the children themselves, parent interviews, and community interviews. Direct observations of the child's social interchanges with an unfamiliar adult, and in some cases, with a parent include the Autism Diagnostic Observation Schedule (ADOS), the Screening Tool for Autism in Two Year Olds, and the Autism Observation Scale. In the ADOS, the gold standard for this kind of assessment, the clinician attempts to engage the child via a proscribed set of activities or questions intended to elicit a variety of behaviors and emotions. Parent interviews include the Autism Diagnostic Interview-Revised (ADI-R) and the Diagnostic Interview for Social and Communication Disorders, designed to obtain information about a child's current and past behaviors. These assessments require highly trained specialists who are ideally ‘calibrated’ against other professionals with similar training. However, there is a shortage of such specialists. Even for the best-served populations (English speaking children in urban areas), waiting times between initial screens in pediatric clinics and autism assessment (ADI-R, ADOS), can exceed 18 months.

Since current assessments for autism are conducted by clinicians, they are subjective, course-grained, and expensive. Clinical staff often bias for a particular diagnosis early in a child's responses to the assessment. Inherently, these assessments may present substantial obstacles for the routine and fine-grained assessments for randomized control trials for autism, for child compliance with the test, and for research purposes. Further, subject motivation is a core challenge for such behavioral assessments.

For example, the Autism-Diagnosis Observational Schedule (ADOS), provides a structure, like a script, that a clinician can use to interview a child. An overall ADOS score may be compiled from approximately 15-20 behavioral scores, each one assigned by subjective observation on a scale from 0 (normal) to 2 (unambiguously impaired). Such a paradigm is sensitive to variations in clinical ability to engage the child and scoring biases. ADOS administration is also expensive, precluding repeated tests of the same child to assess treatment efficacy. The high expense and long waiting times for ADOS assessment preclude large-scale assessments of typically developing (TD) children and atypical children without autism. Further, the ADOS requirement for highly trained clinicians presents clinical barriers. For example, ADOS exams are rarely repeated to assess child improvement in response to therapy and there is often a lengthy wait time between referral and appointment. Such a long waiting time is at cross-purposes with the known benefits of early detection and early intervention. Additionally, ADOS assessments are currently not adequately provided to rural and non-English speaking children because clinicians are primarily English speaking and based in urban clinics. Further, ADOS scores have not currently been validated or calibrated against physiological measures (biomarkers) or measures of brain circuitry, for example. For these reasons, the ADOS remains poorly suited for assessments of intervention and an obstacle to progress in imaging and genetics research, for example.

### SUMMARY

The present disclosure is directed to systems and methods for quantitative assessment of autism spectrum disorder based on detected responses of a subject to displayed animations. Embodiments of the systems and methods described herein utilize various sensors and/or devices to monitor and track responses of a subject to specific animation sequences displayed on a display device or surface. For example, an eye tracking device may be used to detect a subject's eye gaze location on the displayed animation; one or more cameras may be used to capture subject gestures, posture, and/or facial expressions; and/or one or more sensors, e.g., heart rate sensors, respiration sensors, accelerometers, temperature sensors, electroencephalography electrodes, etc., may be used to measure physiological responses and/or motions of the subject while the subject views the animation sequences.

The animation sequences may be specifically selected or generated to display animated characters, objects, event sequences, and/or scenarios used to target specific autism constructs for autism diagnosis. Such autism constructs correspond to autistic behavioral phenotypes such as joint attention, emotion recognition, shared affect, theory of mind, social engagement, narrative, creativity and imagination, imitation, coherence in storytelling, etc. Detected responses to the displayed animations may include eye gaze, facial expressions, gestures, postures, movement, and/or physiological responses such as heart rate, respiration rate, pupil dilation, skin conductance, electrical activity, brain activity, body temperature, and other metrics of arousal. The detected responses may be processed to generate quantitative autism assessment metrics.

In one example approach, a computer-implemented method for performing an autism assessment of a subject may comprise presenting a movie animation on a display and tracking the subject's eye gaze location on the display during specific scenes occurring within the animation, where the specific scenes are associated with an autism construct. An amount of time the subject's eye gaze tracks to predetermined regions on the display within the specific scenes may be calculated to obtain a calculated eye gaze time associated with the specific scenes and the autism construct, and an indication of the calculated eye gaze time may be output. Further, in some examples, a difference between the calculated eye gaze time and an expected eye gaze time associated with the specific scenes and the autism construct may be quantified and an indication of the difference may be output.

Disclosed embodiments may be implemented by a computing system that presents a uniform but highly adaptable series of animated characters, objects, social scenarios, and/or sequences of events to target core constructs of autism. Detected responses to the animation may be processed in real-time to rapidly generate objective, continuous, high resolution measures for several discrete domains of ASD.

Embodiments disclosed herein may be used to provide portable and accessible autism spectrum disorder tests which may be administered at any suitable location, e.g., a familiar environment for a child such as the home, remote locations not readily accessible by clinicians, etc. Further, repeated tests may be easily and inexpensively performed to obtain rapid, objective, fine-grained, and accurate results for autism diagnoses, autism treatment monitoring, and autism research. Further, embodiments may be readily deployed to assess large numbers of typically developing (TD) subjects to generate and fine-tune calibration data and to identify and quantify suitable thresholds and measures of expected responses for various autism constructs, for example.

Because animations are used to target autism constructs, embodiments described herein may be highly adaptable to a specific subject. For example, the animations may be administered in different languages, adapted to different cultural contexts, and/or adapted to any age. In some embodiments, a common library of alterable story elements may be used to accommodate variations in ethnic background or language, for example. Further, such an approach may reduce situational biases associated with clinical autism assessments and may be deployed in a context which promotes motivation of a subject to participate in the test. For example, embodiments disclosed herein may be implemented as an age-specific entertaining animation, a video game, via a website, etc.

Embodiments disclosed herein may utilize animation sequences designed to combine features of storytelling with a display of animated characters, objects, event sequences, and/or scenarios used to target specific autism constructs for autism diagnosis. Storytelling typically engenders some form of empathy from the audience toward a main character, e.g., a protagonist. Social deficits in autism are associated with deficits in empathy, both aspects of cognitive empathy, such as perspective taking and theory of mind, and emotional empathy, which includes shared affect and joint attention. By using storytelling in an assessment, whereby different levels of the subject's social-emotional attachment to various characters presented within a social setting are engendered, a powerful tool for assessments of autism social constructs is provided.

Embodiments disclosed herein may be used to monitor progress of autism treatment and subject development over time to generate progress metrics and assess efficacy of treatment, e.g., embodiments may be used to monitor experimental manipulation of drugs that promote gene transcription for autism treatment. For example, to test a subject repeatedly for the purposes of evaluating treatments, presented animations targeting each construct may be altered to use the same story elements but change characters and scene scenarios. As another example, measures for several discrete domains of ASD generated by embodiments disclosed herein may be compared with brain images or genetic polymorphisms to advance autism spectrum disorder research. Further, in some embodiments, vocalizations may be captured from subjects responding to animated prompts to generate comparison metrics for subjects with ASD to typically developing (TD) and IQ-matched controls for deficits in speech coherence and prosody, for example.

This Summary is provided to introduce a selection of concepts in a simplified form that are further described below in the Detailed Description. This Summary is not intended to identify key features or essential features of the disclosed subject matter, nor is it intended to be used to limit the scope of the disclosed subject matter. Furthermore, the disclosed subject matter is not limited to implementations that address any or all disadvantages noted in any part of this disclosure.

### BRIEF DESCRIPTION OF THE DRAWINGS

FIG. 1 shows a schematic diagram of an example autism spectrum disorder assessment system in accordance with the disclosure.

FIGS. 2-5 show example frames of example displayed animations targeting different autism constructs in accordance with the disclosure.

FIG. 6 shows an example distribution of gaze times in predetermined regions within specific scenes occurring in an animation associated with an autism construct.

FIG. 7 shows an example graph of average gaze times in a predetermined region within specific scenes in an animation associated with an autism construct versus age with example data points for subjects exhibiting an autism condition and subjects not exhibiting an autism condition.

FIG. 8 shows an example graph of average gaze times in regions within scenes occurring within animations associated with different autism constructs with example data points for a subject exhibiting an autism condition and a subject not exhibiting an autism condition.

FIG. 9 shows an example method for performing an autism assessment in accordance with the disclosure.

FIG. 10 schematically shows an example computing system in accordance with the disclosure.

### DETAILED DESCRIPTION

The following detailed description is directed to systems and methods for quantitative assessment of autism spectrum disorder (ASD) based on detected responses of a subject to displayed animations. In the following detailed description, reference is made to the accompanying drawings that form a part hereof, and in which are shown by way of illustration embodiments that may be practiced. It is to be understood that other embodiments may be utilized and structural or logical changes may be made without departing from the scope of this disclosure. Therefore, the following detailed description is not to be taken in a limiting sense. Various operations may be described as multiple discrete operations in turn, in a manner that may be helpful in understanding embodiments; however, the order of description should not be construed to imply that these operations are order dependent and, in some examples, one or more operations described herein may be omitted.

ASD is a complex disability and its characteristics vary enormously from subject to subject. Impairments in social interaction and communication include deficits in joint attention, avoidance of gaze toward eyes, impairments in the ability to perceive emotional information from facial expressions, gestures, or affective intonation in speech, and resulting deficits in empathy. Autistic subjects can also have difficulty with imitation and may be less able to synchronize their gestures with verbal communication or infer emotions and motivations from the actions of characters in a story or relate a coherent narrative. These distinct yet related social deficits may represent distinct and to some extent overlapping diseases, each with their own genetic and environmental etiologies and dissociable patterns of brain activity. If precisely quantified within large cohorts, these distinct behavior phenotypes might associate with discrete genetic polymorphisms or patterns of brain function.

As remarked above, current assessments for autism are conducted by clinicians and are subjective and course-grained. In such approaches, clinical staff may prematurely ‘close’ or focus on a particular diagnosis early in a subject's responses to the assessment. Inherently, these assessments present substantial obstacles for the routine and fine-grained assessments for randomized control trials for autism, for compliance with the test, and for research studies attempting to dissociate genetic or anatomical correlates to variables within the disease. Current assessments for autism are expensive and typically conducted in the English language, thereby presenting obstacles to the assessment of minorities in the US and for standardization across countries. Further, subject motivation is a core challenge for such behavioral assessments.

In order to at least partially address these issues, embodiments of the systems and methods described herein utilize various sensors and/or devices to monitor and track responses of a subject to specific animation sequences selected or generated to target select autism constructs. While a subject views the specific animation sequences, detected subject responses may be processed in real-time to rapidly generate objective, continuous, high-resolution measures for several discrete domains of ASD.

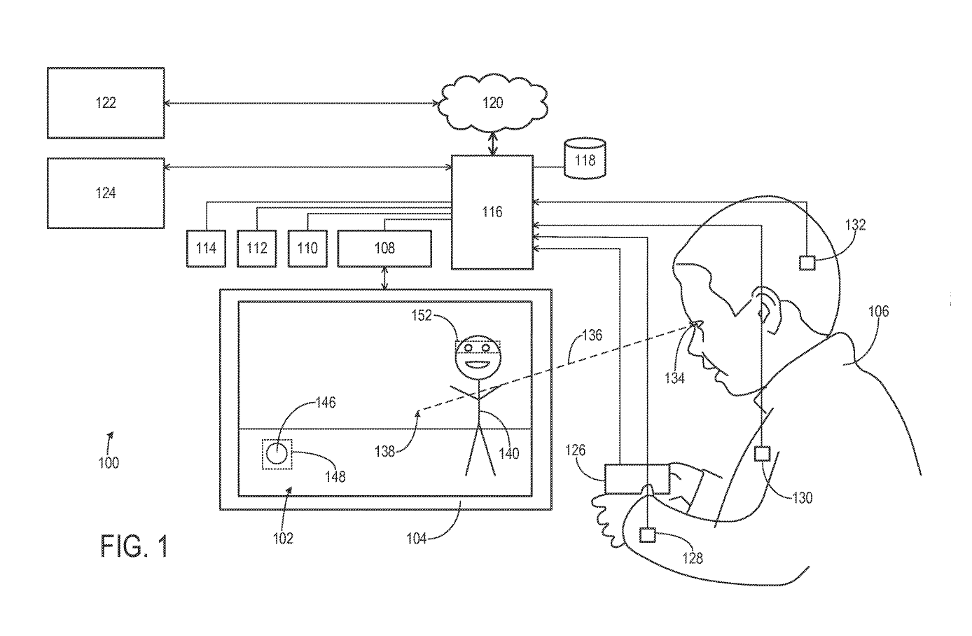

FIG. 1 shows a schematic diagram of an example autism spectrum disorder assessment system 100 for quantitative assessment of autism spectrum disorder based on detected responses of a subject 106 to displayed animations. System 100 includes a display system 108 configured to display animations, e.g., animation 102, on a display 104 viewed by subject 106. Animation 102 may comprise a series of still frames or moving images displayed in succession showing animated characters, objects, event sequences, and/or social scenarios used to target specific autism constructs for autism diagnosis.

Animation 102 may comprise any suitable type of animation such as traditional animation, stop motion animation, computer-generated animation, etc. In some examples, animation 102 displayed on display 104 may be selected or loaded from a database 118 which contains a library of animations, where each animation within the library targets a specific autism construct. Further, in some examples, each animation in the library of animations may be tailored to various subject parameters such as an age range, language, cultural context, IQ range, gender, sex, etc.

Display system 108 may output animations to display 104 utilizing virtually any type of technology. Display 104 may include any suitable display device or surface. Non-limiting examples of display 104 include a computer monitor, television, head-mounted display device, a projection screen or wall, tablet, smart phone, mobile device, etc. Specific examples of display technologies that may be used include cathode ray tube displays (CRT), light-emitting diode displays (LED), electroluminescent displays (ELD), electronic paper (E ink), plasma display panels (PDP), liquid crystal displays (LCD), organic light-emitting diode displays (OLED), optical or video projector systems, etc.

System 100 may include one or more image capture devices 110 configured to collect two-dimensional (2D) and/or three-dimensional (3D) images of subject 106 while subject 106 views animations displayed on display 104. For example, the one or more image capture devices may include one or more cameras configured to capture still photographs and/or moving images such as videos or movies of subject 106. Non-limiting examples of image capture devices that may be included in system 100 include movie cameras, video cameras, internet protocol (IP) cameras, digital cameras, web cameras, depth cameras, stereo cameras, time-of-flight cameras, infrared or near-infrared cameras, optical sensors, etc. As described in more detail below, in some embodiments, images captured by the one or more image capture devices 110 may be used to calculate the target of the subject's eye gaze 136 on display 104 at which one or both eyes 134 of subject 106 are directed and/or focused, i.e., a location 138 on the display 104 where the eye gaze 136 or focus of subject 106 intersects the display 104. Additionally, images captured by the one or more image capture devices may be used to determine motions, facial expressions, gestures, postures, etc. of the subject. Further still, images captured by the one or more image captures devices may be stored in a suitable storage medium, e.g., a tangible computer readable medium, for various processing and/or for analysis by a clinician, for example.

System 100 may also include one or more audio capturing devices 112, e.g., one or more microphones, configured to receive sounds produced by subject 106, e.g., vocalizations and/or speech of the subject. In some examples, sounds captured by the one or more audio capturing devices may be processed to calculate a response of the subject to prompts or stimuli provided to the subject while an animation is displayed on display 104. Further, in some examples, a processor may be included to perform speech recognition on input received from the audio capture devices. Additionally, in some embodiments, system 100 may also include one or more speakers 114 for outputting sounds, e.g., to prompt the subject for input and/or to supplement animations displayed on display 104.

System 100 may include one or more input devices, e.g., input device 126, configured to receive voluntary responses from the subject 106. Non-limiting examples of input devices which may be included in system 100 include keyboards, game controllers, touch screens, manually actuated buttons, switches, mice, etc.

Additionally, system 100 may include various sensors coupled to subject 106, e.g., sensors 128, 130, and 132. In some examples, such sensors may be used to monitor various physiological and/or motion-based responses of the subject to animations displayed on display 104. For example, sensor 128 may comprise one or more accelerometers configured to detect changes in motion of the subject, sensor 130 may comprise a heart rate sensor, respiration sensor, temperature sensor, skin conductance sensor, etc., and sensor 132 may comprise one or more electroencephalography electrodes configured to measure electrical activity. In some examples, sensors coupled to the subject may assist in tracking eye movement and/or eye gaze location of the subject. For example, one or more sensors may be included in contact lenses, eye-glasses, or other head-mounted devices worn by subject 106 to track the eyes of subject 106. As another example, electrodes, magnetic field sensors, and/or mirrors may be placed adjacent to the eyes of the subject to track eye movement.

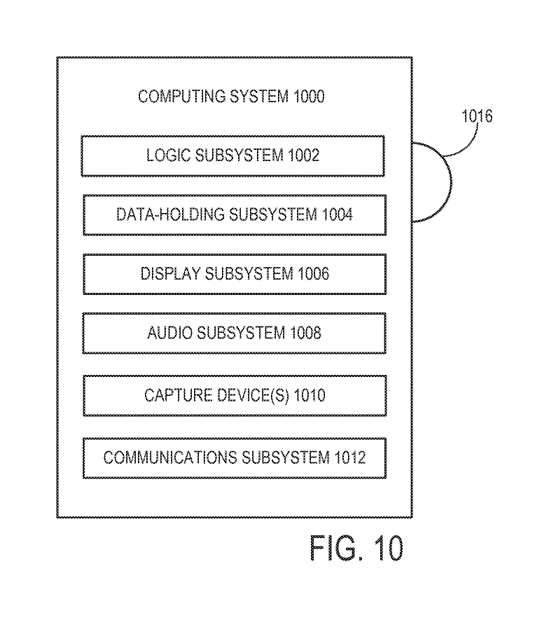

System 100 additionally includes one or more processors 116 in communication with various systems, components, and devices of system 100. The one or more processors 116 may comprise one or more computing systems (generally described below with reference to FIG. 10) which may be in communication with the display system 108, the image capture devices 110, the audio capture devices 112, the speakers 114, the sensors coupled to subject 106 (e.g., sensors 128, 130, 132), the input devices (e.g., input device 126), and various memory components, electronic storage systems, and/or databases (e.g., database 118). The one or more processors 116 may be configured to receive data from one or more sensors and components in system 100 and output data to one or more components of system 100. The one or more processors 116 may include physical circuitry programmed to process data received by the one or more processors and to execute various operations and acts described herein.

For example, the one or more processors 116 may be configured to receive data from one or more sensors and calculate the subject's eye gaze location on the display 104 (i.e., a point of gaze of the subject on display 104) based on the data received from the sensors. The processors may be configured to calculate the subject's eye gaze location on the display in any suitable way. As an example, the processors may be configured to identify a center of the pupil and use infrared/near-infrared non-collimated detected light to detect corneal reflections (CR). A vector between the pupil center and the corneal reflections may be used to compute the point of regard on the display surface or the gaze direction. In embodiments, a bright-pupil or a dark-pupil eye-tracking technique may be used. Further, in some examples, the processor may be configured to detect changes in pupil dilation and correlate pupil dilations with emotional responses. A calibration procedure may be performed to calibrate system 100 for eye-tracking and/or other response monitoring. For example, data collected from the eye-tracking sensors may be synchronized with the animation presented on the display.

As another example, the one or more processors 116 may be configured to detect facial expressions of the subject in one or more images received from the one or more image capture devices 110. Facial expressions may be detected in the images in any suitable way. As an example, the processors may be configured to detect facial landmarks such as the location of the inner and outer corners of the eyes, the tip of the nose, mouth, etc. The processor may be further configured to estimate emotional responses based on the detected facial expressions. Estimating emotional responses may be performed in any suitable way. For example, machine learning may be used to classify detected facial expressions into a set of predetermine emotions such as joy, anger, surprise, fear, sadness, disgust, contempt., frustration, confusion, etc. As another example, detected facial expressions may be classified using a suitable classifier into positive and negative responses. In some examples, system 100 may synchronize detected facial expressions and/or estimated emotional responses with the displayed animation.

As still another example, the one or more processors 116 may be configured to detect gestures, movement, and/or postures of the subject during display of the animation based on data received from sensors in system 100. As one example, data received from accelerometers coupled to the subject, e.g., sensor 128, may be used to calculate motion and/or estimate gestures of the subject. As another example, gestures, movement, and/or postures of the subject may be detected in images received from the one or more image capture devices 110. Gestures, movement, and/or postures may be identified in any suitable way. For example, regions of the subject may be identified in the images and classified using a suitable classifier into different classes of gestures, postures, and/or movement types. As an example, a shape descriptor may be extracted for the region of the subject identified in the images and the shape descriptor may be classified based on training data to estimate the posture, gesture, and/or movement of the region. In some examples, data received from the sensors may be time-stamped and/or may be synchronized with the animation presented on the display.

As yet another example, the one or more processors 116 may be configured to estimate and classify physiological responses based on data received from one or more sensors in system 100. For example, processors 116 may be configured to estimate and/or classify physiological responses based on one or more of a detected heart rate, detected respiration rate, detected body temperatures, and detected electroencephalography data, etc.

One or more processors 116 may be included in one or more devices local to or in proximity with display 104. For example, the one or more processors 116 may be included in a physical component, such as a flash drive, dongle, or other computer product which may be put in communication with a computing system to provide the autism assessment functionality described herein. As another example, the one or more processors 116 may be included in a computing system capable of displaying animations on a surface or display device. As yet another example, the one or more processors 116 may be included on a remote or external computing device or server (e.g., computing device 122 or computing device 124) configured to deliver the autism assessment functionality described herein via a network or other suitable connection. System 100 may be configured to interface with various external or remote computing devices in any suitable manner. For example, remote computing device 122 may be configured to send data to the various components of system 100 via network 120 and/or receive data from various components of system 100 via network 120. For example, computing devices 122 and/or 124 may be used to store data associated with an autism assessment test performed by system 100, to select or generate animations for display on display 104, to specify and send prompts to system 100, to monitor progress of an autism assessment test performed by system 100, etc.

The animation 102 displayed on display 104 may be specifically selected or generated to display animated characters, objects, event sequences, and/or scenarios used to target specific autism constructs for autism diagnosis. As remarked above, the autism constructs correspond to autistic behavioral phenotypes such as joint attention, emotion recognition, shared affect, theory of mind, social engagement, narrative, creativity and imagination, imitation, coherence in storytelling, etc. Responses detected by system 100 may be processed to generate quantitative autism assessment metrics relative to predetermined thresholds or expected responses.

In some embodiments, animated stories may be used to assess autistic children through the power of narration, a process that begins with setting an empathic feeling in the spectator for a particular character (e.g., a protagonist) and then setting that character on a journey that entails progressively more difficult situations. For example, by quantifying a disparity in a subject's level of empathy for one character versus another through a presented narrative process, e.g., by using different characters (protagonist versus a more incidental character) as animated stimuli, core constructs of autism may be assessed. As another example, social tension may be generated in a presented story and a subject's response may be tracked to provide a measure to assess a subject's social interest under increasing levels of interest Animations presented to the subject may be generated to amplify this tension, for example. Additionally, various distractors may be included in a displayed animation to draw attention away from a primary social scene. Monitoring a subject's response to increases and decreases in tension and distractors presented in an animation provides data from which quantitative metrics for autism assessment may be extracted.

For example, animation 102 shown in FIG. 1 displays a scene with a character 140 and an object 146 located next to the character 140. The animated scene displayed on display 104 comprises a sequential series of still-image frames displayed in succession to create an illusion of continuous motion or shape change. While the animation 102 is displayed on display 104, the subject 106 may freely view the displayed animation while various sensors and components of system 100 detect, monitor, and track responses of the subject 106 to the animation. For example, during display of the animation, the subject may look at or focus on different locations on the display. As described above, system 100 is configured to detect the target of the subject's eye gaze on the display. For example, as shown in FIG. 1, the subject 106 is looking at a point 138 on the display device. System 100 can detect and calculate this eye gaze location in real-time during display of the animation to thereby track where the subject is looking on the display during the animation.

The animations displayed on display 104 comprise a plurality of frames which present different scenes or scenarios designed to target specific autism constructs. In particular, each scene presented may be designed to elicit a particular expected response from the subject, e.g., a particular expected emotional response, a predetermined expected redirection of attention or eye gaze location of the subject, etc.

As one example, during specific times and for specific durations in an animation displayed on display 104, an expected response of the subject for a given autism construct may comprise the subject looking at a particular region of the display, e.g., a particular object or a particular character displayed in the animation. Thus, predetermined eye gaze regions may be specified in a set of pre-selected frames of the animation where the set of preselected frames is associated with a specific autism construct. For example, as a measure of the subject's ability for joint attention, during a set of preselected frames in animation 102 a predetermined region 148 around object 146 may be specified and system 100 may be configured to calculate an amount of time the subject's eye gaze location on the display is within this predetermined region 148. For example, beginning at a specified time in the animation and for a specified duration, object 146 may be identified as a salient object and a region 148 on display 104 bounding object 146 may be specified. While object 146 is identified as a salient object, system 100 may continuously track the subject's eye gaze location on the display and calculate an amount of time that the subject's eye gaze is within region 148. The amount of time the subject's eye gaze location on the display is within this predetermined region 148 may then be compared with responses of other subjects, e.g., typical subjects, subjects with other disabilities or with matching IQ, and/or with other subjects with autism. In some examples, the amount of time the subject's eye gaze location on the display is within this predetermined region 148 may be compared against different kinds of data acquired from typically developing (TD) subjects.

Various other parameters may be extracted from eye tracking data while a subject views the animation. For example, latency between the time that the character turns its eyes (region 152) to look toward the object 146 and the time that the subject looks at the object 146 may be calculated. As another example, a number of back and forth movements of the subject's eye gaze toward the object of the character's attention and the eyes of the character may be calculated. The parameters extracted from eye gaze data may be compared to control parameters to generate metrics for autism assessment. For example, the amount of time the subject's eye gaze location on the display is within the predetermined region 148 may be compared with averages for autistic, typical, and/or atypical but not autistic children to provide a metric for autism assessment.

As another example, animation 102 may display character 140 speaking or gesturing and an expected gaze of a TD subject may be at the eyes of the character within region 152 on display 104. In this example, system 100 may continuously track the subject's eye gaze location on the display and calculate an amount of time that the subject's eye gaze is within region 152 while the character is speaking or gesturing. The amount of time the subject's eye gaze location on the display is within this predetermined region 152 may then be compared with an expected eye gaze time, e.g., with averages for autistic, typical, and/or atypical but not autistic children, to provide a metric for autism assessment.

In some examples, the animations displayed by system 100 may be selected or generated from a library or database of animations or scenes, e.g., stored in database 118. Such a database of animations may include various pre-created animation sequences associated with different autism constructs and with specified salient regions at certain times and durations in the animation sequences. The animations may be selected, adjusted, or generated based on various parameters associated with the subject. For example, the animations may be selected, adjusted, or generated based on an age of the subject, first language of the subject, gender or sex of the subject, an IQ of the subject, cultural context of the subject, etc. In some examples, before displaying an animation to assess autism, system 100 may prompt a user for input to obtain subject parameters, and/or may provide a suitable test for estimating an IQ of the subject. Additionally, various calibration operations may be performed prior to initiating an autism test, e.g., calibrations of various sensors, calibrations for facial recognition, eye tracking, etc.

In some examples, the animation displayed on display 104 may include different scenes or frames that repeatedly target a given autism construct so that average response results for the given autism construct can be calculated. Additionally, the animation displayed may include different scenes or frames which target multiple different autism constructs so that an autism assessment can be performed based on response results across multiple different autism constructs as described in more detail below with regard to FIG. 8.

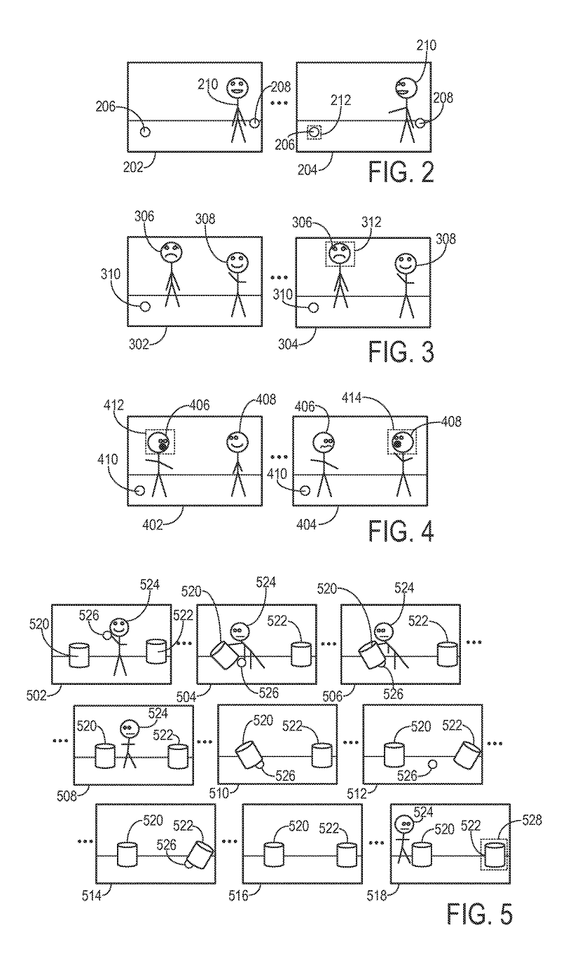

FIGS. 2-5 show example scenes of example displayed animations which target different autism constructs. During display of these example animation sequences, responses of a subject viewing the animation sequences may be detected and processed to quantify responses of the subject relative to expected responses. It should be understood that the example scenes shown in FIGS. 2-5 are provided for illustrative purposes and are not intended to be limiting.

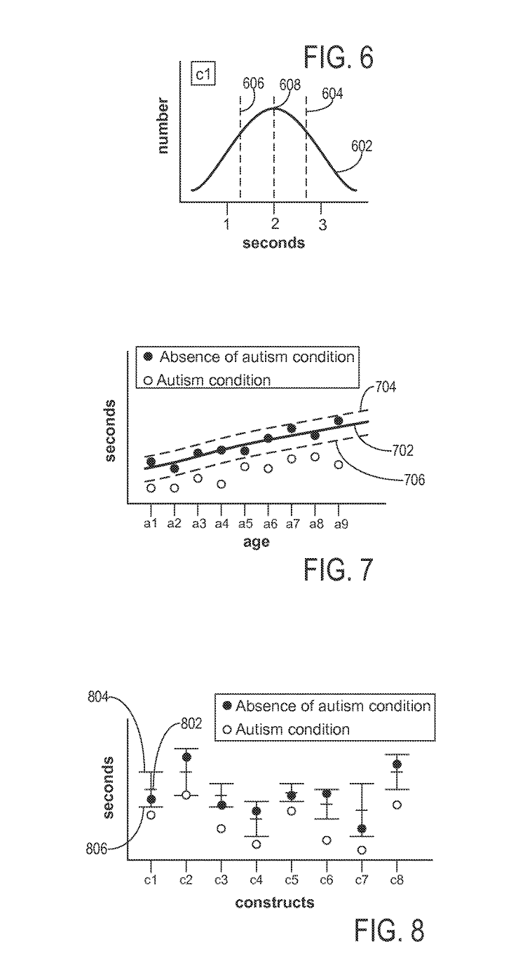

FIG. 2 shows a first sequence of frames 202 followed by a second sequence of frames 204 of an animated scene which could be used to target one or more autism constructs. In this example, the first sequence of frames 202 of the animation shows a character 210 in a room with an object 206. The second sequence of frames 204 shows the character 210 gesturing or pointing toward the object 206. In the second sequence of frames 204, a region 212 has been specified around object 206 in each frame in the second sequence of frames 204. When this second sequence of frames 204 is viewed by a subject, system 100 detects the subject's eye gaze location on the display and calculates an amount of time the subject's eye gaze location on the display is within region 212. Such an animation sequence may be used to target a joint attention autism construct, for example. In particular, the example animation shown in FIG. 2 may be used to determine if the subject's gaze follows the attention of the character 210. The amount of time the subject's eye gaze location is within region 212 may be compared to an expected eye gaze time, where the expected eye gaze time may be based on response data obtained from control subjects to the animation shown in FIG. 2. As an example, control subjects may comprise one or more of autistic, typical, and/or atypical but not autistic children. For example, the animation shown in FIG. 2 may be displayed to one or more TD subjects and the amounts of time the TD subjects' eye gaze locations are within region 212 may be calculated to obtain a distribution of amounts of time control subjects' eye gaze locations are within region 212 (an example distribution is illustrated in FIG. 6 described below).

FIG. 3 shows a first sequence of frames 302 followed by a second sequence of frames 304 of another example animated scene which may be used to target one or more autism constructs. In this example, the first sequence of frames 302 of the animation shows a first character 306 exhibiting a first emotion (e.g., sadness) in a room with an object 310 and a second character 308, e.g., a protagonist, exhibiting a second emotion (e.g., happiness). The second sequence of frames 304 continues to show the first character 306 exhibiting the first emotion and the second character 308 exhibiting the second emotion. In some examples, between the first sequence of frames 302 and the second sequence of frames 304, system 100 may initiate a predetermined expected redirection of the subject's eye gaze toward a particular character or emotion represented by the character; e.g., system 100 may initiate a predetermined expected redirection of the subject's eye gaze to the character exhibiting the first emotion. The predetermined expected redirection of the subject's eye gaze to the character exhibiting the first emotion may be initiated in any suitable way. For example, the system may prompt the user, via a visual indication or an audio indication, to gaze on the emotionally salient character rather than others in the scene, e.g., the subject may be prompted by a displayed animated character or a narrator to look for a particular emotion. In the second sequence of frames 304, a region 312 has been specified around the face of character 306 in each frame in the second sequence of frames 304. When this second sequence of frames 304 is viewed by a subject, system 100 detects the subject's eye gaze location on the display and calculates an amount of time the subject's eye gaze location on the display is within region 312. The animation sequence shown in FIG. 3 may be used to target an emotion recognition autism construct, for example. In particular, the example animation shown in FIG. 3 may be used to determine if the subject's gaze is directed to an emotionally salient character in an animated scenario.

In some examples, a group of displayed animated characters may present different emotions or a character expressing an emotion (e.g. joy, sadness, fear, startled) may be presented within the context of a larger social situation. In these examples, the subject may be prompted by a character or narrator to look for a particular emotion, e.g., a mother in the displayed scene may say: “My children are always happy” while the animation presents two neutral children and one happy child. TD subjects may be expected to focus on the emotionally salient character rather than others in the scene. However, subjects with an autism condition may gaze on the emotionally salient character for a shorter duration than a TD subject. Additionally, in response to changes in a character's emotional state, facial expressions of a subject with an autism condition may be less pronounced and/or have longer response latencies than TD subjects. Further, subjects with an autism condition may gaze on other objects, characters or regions in the scene while TD subjects focus on the emotionally salient character.

As an example, FIG. 3 shows a third sequence of frames 331 which shows character 306 along with additional characters 341 and 343 which are directing their attention to character 308, e.g., ganging up on character 308. In the third sequence of frames 331, the facial expression of character 308 has changed in response to the displayed interaction with the other characters 306, 341, and 343. In this example, a region 353 is specified around the face of character 308 to track the eye gaze of a subject viewing the animation and to calculate an amount of time the subject's eye gaze is within region 353.

In the example shown in FIG. 3, the amount of time the subject's eye gaze location is within region 312 and/or region 353 may be compared with an expected eye gaze time, where the expected eye gaze time is based on a distribution of eye gaze responses by control subjects to the animation. The control subjects may comprise one or more of TD subjects, atypically developing but not autistic subjects, and autistic subjects. For example, the animation shown in FIG. 3 may be displayed to one or more TD subjects and the amounts of time the TD subjects' eye gaze locations are within region 312 and/or region 353 may be calculated to obtain a distribution of amounts of time control subjects' eye gaze locations are within region 312 and/or region 353 (see FIG. 6 described below).

Though the example shown in FIG. 3 shows only one specified region in a sequence of frames, e.g., region 312 or region 353, for tracking a subject's eye gaze, in some examples, a plurality of regions may be specified at different locations within specific scenes occurring in an animation and an amount of time the subject's eye gaze tracks to these regions may be calculated. For example, an amount of time the subject's eye gaze is in a group of regions may be calculated and compared to an expected amount of time. In this way, durations of eye gaze within a particular group of boxes of interest (like region 312) for each face and potentially each object that competes for subject attention due to its color, shape, or movement, may be calculated and compared with the distribution of amounts of time typical, autistic, and/or atypical non-autistic subjects' eye gaze locations are within the particular group of boxes. As another example, a number of transitions between a first region and a second region may be calculated and compared to an expected number of transitions during the assessment.

FIG. 4 shows a first sequence of frames 402 followed by a second sequence of frames 404 of another animated scene which could be used to target one or more autism constructs. In this example, the animation sequence shows a first character 406 engaging in an exchange, e.g., conversing or interacting, with a second character 408. The first sequence of frames 402 of the animation shows the first character 406 exhibiting an active behavior, e.g., speaking to the second character 408, where the second character is exhibiting a passive behavior, e.g., listening. The second sequence of frames 404 shows the second character 408 exhibiting an active behavior, e.g., speaking to the first character 406, while the first character 406 exhibits a passive behavior, e.g., listening. In both frame sequence 402 and frame sequence 404, regions 412 and 408 have been specified around the face of each character. For example, the animation sequence shown in FIG. 4 may be used to target a social engagement autism construct to determine if the subject's eye gaze follows a back and forth exchange, e.g., a conversation, between the animated characters.

When the animation shown in FIG. 4 is viewed by a subject, system 100 may detect the subject's eye gaze location on the display and calculate an amount of time the subject's eye gaze location on the display is within regions 412 and 414. Various parameters may be calculated based on the amount of time the subject's eye gaze location on the display is within regions 412 and 414. For example, durations of eye gaze in the targets 412 and 414 may be calculated and/or the number of shifts in glance between target regions 412 and 414 may be calculated. Additionally, changes in subject facial expression accompanying changes in emotion of either character or in response to a change in tension between characters may be detected, and eye gaze toward objects that compete for subject attention, e.g., due to its color, shape, or movement, may be calculated and compared with expected responses.

FIG. 5 shows another example animation sequence which may be used to target one or more autism constructs. For example, the animation sequence shown in FIG. 5 may be used to target a theory of mind (ToM) construct. In particular, the animation sequence shown in FIG. 5 may be used to determine if a subject viewing the animation understands that an animated character can harbor a false belief. The animation sequence shown in FIG. 5 comprises a first sequence of frames 502 which shows a character 524 holding an object 526 and a first bucket 520 and a second bucket 522 in the displayed scene. A second sequence of frames 504 and a third sequence of frames 506 show character 524 placing object 526 in the first bucket 520 so that the object is hidden inside the first bucket 520 as shown in the fourth sequence of frames 508. In the fifth sequence of frames 510, sixth sequence of frames 512, seventh sequence of frames 514, and eighth sequence of frames 516, the character 524 has left the room and the object 526 is shown being moved from beneath the first bucket 520 to be hidden beneath the second bucket 522 while out of view of the character 524. In the ninth sequence of frames 518, the character 524 returns to the room. In some examples, during the ninth sequence of frames, system 100 may initiate a predetermined expected redirection of the subject's eye gaze to the location of the hidden object. The predetermined expected redirection of the subject's eye gaze to the hidden object may be initiated in any suitable way. For example, the system may prompt the user, via a visual and/or audio indication, to identify where character 524 thinks object 526 is hidden. In response, the subject may provide input, e.g., via an input device or by providing speech or gesture input, to indicate where character 524 thinks object 526 is hidden. In some examples, the target of the subject's eye gaze may be used as subject input indicating where character 524 thinks object 526 is hidden. For example, in the ninth sequence of frames 518, a region 528 may be specified around the second bucket 522 where object 526 is hidden and/or a region 561 may be specified around bucket 520 where object 526 is not hidden. When this ninth sequence of frames 518 is viewed by a subject, system 100 may detect the subject's eye gaze location on the display and calculate an amount of time the subject's eye gaze location on the display is within region 528 and/or region 561. As with measures employed to assess the above-described constructs, the amount of time the subject's eye gaze location is within region 528 and/or 561 may be calculated and compared with parameters extracted from control distributions, e.g., a distribution of amounts of time typical, autistic, and/or atypical non-autistic subjects' eye gaze locations are within regions 528 and/or 561.

Various other responses of the subject may be detected while a subject views the example animation sequences shown in FIGS. 2-5. For example, system 100 may detect input by the subject via one or more input devices, e.g., input device 126. System 100 may detect facial expressions, gestures, postures, and/or movement from one or more images of the subject received by the system. System 100 may detect various physiological responses of the subject while the subject views the animation sequences. These detected responses and inputs may be used quantify the subject's responses for various autism constructs.

As one example, a subject's emotional response, salience, or tone may be estimated based on images and/or sensor data received by system 100 while the subject views an animation. The estimated emotional salience or response may be compared to an expected emotional salience or response to determine whether the subject's emotions are responsive and/or appropriate to the emotional state or situation of the characters and/or scenes displayed. For example, to target a shared affect autism construct, system 100 may assess synchrony between the subject's estimated emotional response and a displayed character's emotional expression or physical situation. The subject's emotional response may be estimated from one or more of detected facial expressions, detected physiological responses, and detected gestures, postures, or motions of the subject. The estimated emotional response may then be compared to an expected emotional response for a displayed scene in order to generate a score for a shared affect autism construct. Such expected emotional responses may be based on emotional responses of TD subjects estimated from detected responses of the TD subjects while they view the animation. In some examples, a difference between the subject's estimated emotional response and the expected emotional response may be calculated, and the system 100 may output an indication of the calculated difference. As an example, such indications output by system 100 may comprise a visual indication output to a display device that visually indicates a difference between the estimated response and an expected response. As another example, such indications output by system 100 may comprise a calculated score that quantifies a difference between the estimated response and an expected response. Such scores may be saved in a storage medium (e.g., an electronic, non-transitory storage medium or a memory component of a computing device) and, in some examples, saved in a specific format, e.g., as a table or list in a spreadsheet, as data in an XML document, as data in a database, etc.

As another example, system 100 may target a narrative construct by prompting the subject to recount what happened during a displayed sequence of events (an animated story) after the sequence of events was viewed by the subject. For example, the system 100 may prompt the subject to provide input, e.g., speech input, gesture input, eye gaze input, etc., to assess the subject's understanding of what happened (e.g., the narrative) during the displayed sequence of events. For example, system 100 may prompt the subject to recount the events of the story. In some examples, video of the subject's response may be recorded by an image capture device and/or audio of the subject's response may be recorded by an audio capture device. The audio and/or video recordings may be stored in a storage medium, e.g., an electronic storage medium or a memory component of a computing device. Assessment of the subject's verbal response may be processed manually and/or by speech recognition algorithms to assess frequency and amplitude modulation (affective prosody), frequency and amplitude modulation in synchrony with syntax and/or the meanings of specific words (grammatical or pragmatic prosody), pronunciation, word selection, and story cohesion. For example, recognized speech in the audio input may be used to evaluate how well the subject used language that indicates the emotions and motivations of the characters displayed in an animation. For example, unusual word usage outside the relevance of the presented story narrative may be identified. As another example, system 100 may present a series of questions, e.g., via display 104 or via speakers 114, to ascertain the subject's understanding of the sequence of events. System 100 may receive the subject's responses to the questions and calculate a score representing the subject's understanding of the sequence of events. Such scores may be compared to scores obtained from control subject responses, e.g., TD subjects' responses.

As still another example, system 100 may target a creativity and imagination construct by prompting the subject to describe and/or envision what might happen subsequent to a displayed sequence of events, e.g., if the story were to continue. In some examples, audio and/or video recordings of the subject's response may be collected by microphones and/or image capture devices. The collected audio and/or video recordings may be stored in a storage medium, e.g., an electronic storage medium or a memory component of a computing device. In some examples, detected audio input may be processed by a speech recognition algorithm and used to evaluate how much of the response is related to socially and emotionally relevant information versus details about surrounding objects and information not represented within the animation, for example.

As yet another example, system 100 may target an imitation autism construct by prompting the subject to mimic or reproduce an action performed by an animated character displayed on display 104. For example, system 100 may display an animation which shows a character performing an action and may prompt the subject to imitate the action performed by the character. In this example, system 100 may detect gestures, postures, facial expressions, and/or movements, e.g., via accelerometers, movie images captured of the subject, etc., to determine how accurately the subject imitates the action. Various parameters may be extracted from the subject's response to the displayed action and used to calculate an imitation score that may be compared with imitation scores of typical, autistic, and/or atypical non-autistic subjects. For example, one or more of response latency, accuracy of movement, timing of the movements relative to one another, accelerometer data, etc., may be extracted from various sensors in system 100 and used to calculate a measure of the subject's response for the imitation autism construct.

FIG. 6 shows an example distribution 602 of gaze times in a predetermined region in a set of frames in an animation associated with a construct c1. Construct c1 may comprise any suitable autism construct (examples of which are described herein). It should be understood that the example distribution 602 shown in FIG. 6 is provided for illustrative purposes and is not to be taken in a limiting sense. Though, FIG. 6 shows a normal distribution, such distributions may take any suitable form, e.g., bimodal or multimodal.

In some examples, a threshold for each construct may be used to compare a subject's specific response to a displayed animation targeting the construct to a control subject's responses to the animation. For example, the subject's response to the animation may be compared with a mean (average) or median response of measured control subjects to the specific animated scenes targeting a specific construct. For example, the animation associated with the autism construct may be displayed to one or more TD subjects and the amounts of time the TD subjects' eye gaze locations are within the predetermined region may be calculated to obtain the distribution 602 of amounts of time control subjects' eye gaze locations are within the predetermined region. Any suitable number of control subjects may be used to generate such a distribution, e.g., 1 control subject, 10 control subjects, 1000 control subjects, etc. Further, the control subject data used to generate the distribution may be obtained from any suitable control subject population. As one example, the control subjects may comprise typically developing subjects. As another example, the control subjects may comprise atypically developing but not autistic subjects (e.g. children with ADHD, Down's syndrome, etc.). As still another example, the control subjects may comprise autistic subjects.

In this example, the distribution is a normal distribution and the mean 608 corresponds to the peak of distribution 602. This average value obtained from the distribution may be used to quantify a difference between a subject's score for a measure of construct cl in the context of autistic and control populations. For example, if the amount of time a subject's eye gaze targets the predetermined region falls below a standard deviation 606 of the distribution, then an autism condition for the autism construct may be indicated. As another example, if the amount of time a subject's eye gaze location is within the predetermined region is within an interval around the average 608 of the distribution, e.g., between the average minus a standard deviation 606 and the average plus the standard deviation 604, then an absence of an autism condition for the autism construct may be indicated.

In some examples, distributions, such as distribution 602, may be generated for other parameters extracted from detected subject responses to a displayed animation. Examples of such parameters may include duration of eye gaze within a specified region, response latencies, degree and relevance of facial expression changes in response to animated social situations, story coherence, prosody, gestural imitation, etc.

The distributions for autism constructs may depend on various subject parameters such as age, gender, sex, IQ, cultural context, etc. For example, the distribution 602 for the autism construct c1 may be different at different ages, e.g., the average 608 and standard deviations of distribution 602 may be different for different age groups. FIG. 7 shows an example growth chart that illustrates different distribution averages 702 at different ages (a1, a2, a3, a4, a5, a6, a7, a8, a9) for a given autism construct. In FIG. 7, the average values 702 correspond to the averages of distributions, e.g., average 608 of distribution 602, calculated for TD subjects with different ages. The average 702 is bounded above by the average plus the standard deviation of the distribution for a given age (indicated by curve 704) and bounded below by the average minus the standard deviation of the distribution for the given age (indicated by curve 706). It should be understood that though FIG. 7 shows averages increasing with increasing age, in some examples, averages may decrease with increasing age, remain substantially constant with increasing age, or increase and decrease across different ages.

For each age, FIG. 7 also shows example calculated gaze times for subjects exhibiting an autism condition for the given autism construct (indicated on the graph by gaze time data points below curve 706) and subjects exhibiting an absence of an autism condition for the given construct (indicated on the graph by gaze time data points between curves 704 and 706).

The gaze time distributions, e.g., distribution 602 shown in FIG. 6, may be calculated for a plurality of constructs and used to generate a multi-factor autism assessment map as shown in FIG. 8. Such a multi-factor autism assessment map may be used to quantify a subject's response mapping across a plurality of autism constructs. In particular, FIG. 8 shows typical control gaze times and intervals for different autism constructs (c1, c2, c3, c4, c5, c6, c7, and c8). For example, for construct cl, the average gaze time is shown at 802 and the interval is bounded above by 804 and below by 806. For example, the average gaze time 802 may correspond to average 608 obtained from the distribution 602, upper threshold 804 may correspond to point 604 on distribution 602, and lower threshold 806 may correspond to point 606 on distribution 602.

For each construct shown, FIG. 8 shows example calculated gaze times for a subject exhibiting an autism condition (indicated on the graph by gaze time data points below lower thresholds of the intervals, e.g., below lower threshold 806 for construct c1) and a subject exhibiting an absence of an autism condition (indicated on the graph by gaze time data points between upper and lower thresholds of the intervals, e.g., between upper threshold 804 and lower threshold 806 for construct c1).

Such multi-factorial assessment maps may be used to identify patterns across multiple autism constructs, e.g., via principal components analysis, classification and regression tree (CART) analysis, and/or K means clustering. In this way, many related disorders that share similarities but differ at a fine-scale level may be assessed. For instance, there may be a group of subjects, e.g., 10%, that express poor responses to joint attention, emotion recognition and imitation, but fair well on a theory of mind task and story coherence. Another group of subjects, e.g., 20%, may express a different pattern of responses to the autism assessment, with poor performance on joint attention, theory of mind, and facial expression/gestural synchrony while recounting an animated story; yet express the competence of typical children with affective prosody and emotion recognition. A multi-factorial assessment map such as shown in FIG. 8 may be utilized to identify these patterns, to classify subjects that share an ASD diagnosis, and to evaluate ASD for each of its spectrum disorders for genetic and anatomical correlates, for example.

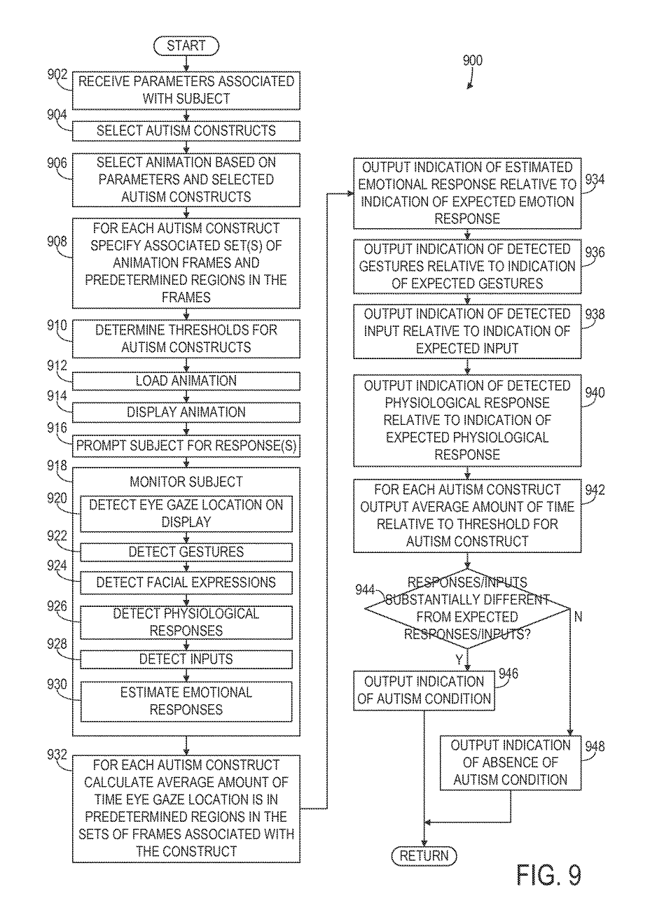

FIG. 9 shows an example method 900 for performing an autism assessment of a subject. Method 900 may be implemented using system 100 shown in FIG. 1 described above. The various acts of method 900 may be performed in the sequence illustrated, in other sequences, in parallel, or in some cases omitted. Likewise, the order of the various acts of method 900 may be changed.

At 902, method 900 includes receiving parameters associated with a subject. For example, a user of system 100 may provide input via a suitable input device to indicate various parameters of the subject. In some examples, system 100 may provide prompts, e.g., visual or audio prompts, to the subject or another user requesting input to select parameters so that the autism assessment test can be adjusted accordingly. Examples of subject parameters include age of the subject, gender of the subject, a first or native language of the subject, a cultural context of the subject, an IQ of the subject, a condition of the subject (e.g., typically developing, atypically developing but not autistic, or autistic), etc. In some examples, system 100 may administer an IQ test, e.g., a non-verbal IQ test such as the Raven's test, to the subject prior to implementing the autism assessment test. Additionally, system 100 may implement various calibration routines, e.g., to calibrate system 100 for eye-tracking, facial recognition, and other response monitoring.

At 906, method 900 may include selecting, adjusting, or generating an animation based on the subject parameters and selected autism constructs. For example, an animation may be selected or adjusted to specifically target selected autism constructs for a specific age, gender, sex, and/or IQ of the subject. As remarked above, in some examples, system 100 may include a database of animation scenes tailored to target specific autism constructs and subject parameters. For example, animations with specific characters, social situations, story length, languages, and/or complexity of background may be selected or generated.

At 908, method 900 includes, for each autism construct, specifying specific scenes and predetermined regions within the scenes associated with the autism construct. The predetermined regions of specific scenes or specific frames occurring within the animation may be specified to track amounts of time a subject's eye gaze is within the predetermined regions while the subject views the animation. The predetermined regions in the frames may be specified in any suitable manner. As one example, a user may select one or more still images of the animation and select a region within each still image. As another example, in a moving scene of the animation, a user may select a region, e.g., a box, around an area of interest and designate a time interval, e.g., 3-10 seconds, which informs system 100 to calculate eye gaze duration in that region for the time interval.

At 912, method 900 includes loading the animation. For example, the selected animation may be loaded into a memory component of a display system, e.g., display system 108. At 914, method 900 includes displaying the animation on a display. For example, display system 108 may present a movie animation on display 104 so that the animation may be freely viewed by a subject.

The animation displayed to the user may comprise a plurality of specific scenes, e.g., sets of pre-selected frames, associated with a plurality of selected autism constructs. As an example, while displaying a set of frames or specific scenes of an animation targeting a joint attention autism construct, presenting a movie animation on a display may comprise presenting animated content on the display to initiate a predetermined expected redirection of the subject's eye gaze to the predetermined regions on the display within the specific scenes. For example, the animated content displayed may comprise an animated character gesturing toward an animated object in the predetermined regions.

As another example, while displaying a specific scene targeting an emotion recognition autism construct, displaying an animation on the display may comprise displaying at least a first character and a second character, where the first character exhibits a first emotion and the second character exhibits a second emotion. In this example, the system 100 may initiate a predetermined expected redirection of the subject's eye gaze to a character exhibiting the first emotion and the predetermined regions may correspond to a location of the first character on the display.

As still another example, while displaying a specific scene targeting an emotion recognition autism construct, presenting a movie animation on a display may comprise displaying a primary character exhibiting an emotion in reaction to a scenario and the predetermined regions may correspond to a location of the primary character on the display.

As yet another example, while displaying specific scenes targeting a theory of mind autism construct, presenting a movie animation on a display may comprise displaying an animated character viewing an object being hidden at a first location then displaying a movement of the hidden object out of view of the character to a second hidden location. In this example, system 100 may initiate a predetermined expected redirection of the subject's eye gaze to the hidden object and the predetermined regions may correspond to the second hidden location of the object.

As still another example, while displaying specific scenes targeting a social engagement autism construct, presenting a movie animation on a display may comprise displaying at least a first animated character and a second animated character or object, where the first character exhibits an active behavior (e.g., speaking) and the second character or object exhibits a passive behavior, and the predetermined regions include regions on the display corresponding to a location of the first character on the display. In this example, presenting a movie animation on a display may further comprise displaying the first character exhibiting a passive behavior and the second character or object exhibiting an active behavior and the predetermined regions may correspond to a location of the second character or object on the display. Further, in these examples, the predetermined regions may correspond to a location of the eyes of the character or object exhibiting an active behavior and/or the locations of distractors displayed in the animation.

At 916, method 900 may include prompting the subject for responses. In particular, while displaying the animation, system 100 may prompt the subject for various inputs or responses. For example, audio or visual prompts may be provided by system 100 to query the subject during pre-selected animated still frames and/or animated movies. As an example, while displaying a set of frames targeting a narrative autism construct, presenting a movie animation on a display may comprise displaying a sequence of events on the display and, following display of the sequence of events, system 100 may prompt the subject for input describing what happened during the sequence of events. As another example, after displaying pre-selected animated still frames and/or animated movies to target a creativity and imagination construct, system 100 may prompt the subject for input describing events subsequent to a displayed sequence of events. As yet another example, presenting a movie animation on a display may comprise displaying a character performing an action and system 100 may prompt the subject to imitate the action performed by the character, detect the subject's action following the prompt, and compare the detected subject's action to the action performed by the character.

At 918, method 900 includes monitoring the subject while the subject views the displayed animation. The subject may be monitored by a variety of sensors, input devices, image capture devices, and audio capture devices that communicate data within or to system 100 for processing. For example, at 920, method 900 may include detecting the subject's eye gaze location on the display during scenes in a plurality of scenes, wherein each scene in the plurality of scenes is associated with an autism construct in a plurality of autism constructs. As another example, at 922, method 900 may include detecting gestures, postures, and/or movements of the subject during the animation. As still another example, at 924, method 900 may include detecting facial expressions. For example, facial expressions of the subject may be detected in one or more images of the subject received by system 100 while the subject views the animation. In some examples, system 100 may record the facial expressions of the subject in synchrony with the subject's viewing of the displayed scenes. As still another example, at 926, method 900 may include detecting physiological responses of the subject during the animation. For example, detecting physiological responses of the subject may comprise one or more of detecting a heart rate of the subject, detecting a respiration rate of the subject, detecting a temperature of the subject, and detecting electrical activity of the subject. As still another example, at 928, method 900 may include detecting input by the subject, e.g., via an input device such as input device 126 shown in FIG. 1. System 100 may be configured to process data received while monitoring the subject to obtain measures of subject responses to the animation. For example, at 930, method 900 may include estimating emotional responses or an emotional salience of the subject. For example, emotional responses may be estimated based on detected facial expressions, detected gestures, postures, or movement, and/or detected physiological responses.

At 932, method 900 includes, for each scene in the plurality of scenes, calculating an amount of time the subject's eye gaze location on the display is within predetermined regions in the scene and associating the calculated amount of time with the autism construct associated with the scene. Additionally, at 932, method 900 may include, for each autism construct in the plurality of autism constructs, calculating an average amount of time associated with the autism construct from the amounts of time associated with the autism construct.

At 934, method 900 includes quantifying differences between the detected subject's responses and expected responses. For example, a difference between the calculated eye gaze time and an expected eye gaze time associated with the specific scenes and the autism construct may be calculated. In this example, the expected eye gaze time associated with the specific scenes and the autism construct may be based on a distribution of eye gaze responses by control subjects to the specific scenes. As one example, the control subjects used to generate such a distribution may comprise typically developing subjects. As another example, the control subjects used to generate such a distribution may comprise atypically developing but not autistic subjects. As still another example, the control subjects used to generate such a distribution may comprise autistic subjects. In some examples, such a distribution may be selected based on one or more of age, sex, gender, and IQ of the subject.

As another example, a difference between an estimated emotional salience of the subject and an expected emotional salience associated with the specific scenes and the autism construct may be quantified. In this example, the expected emotional salience associated with the specific scenes and the autism construct may be based on a distribution of emotional responses by control subjects to the specific scenes. As still another example, a difference between detected gestures of the subject and expected gestures associated with the specific scenes and the autism construct may be quantified. As yet another example, a difference between detected physiological responses of the subject and expected physiological responses associated with the specific scenes and the autism construct may be quantified.

At 936, method 900 may include outputting indications. Various indications may be output based on the detected responses of the subject while viewing the animation. Indications may be output in any suitable way. For example, indications output by system 100 may comprise visual indications output to a display device that visually indicate differences between estimated responses and expected responses. As another example, indications output by system 100 may comprise audio and/or haptic indications output to one or more speakers or components capable of vibration. As another example, such indications output by system 100 may comprise indications of calculated scores which quantify differences between estimated responses and expected responses. Such scores may be saved in a suitable storage medium and, in some examples, saved in a specific format, e.g., as a table or list in a spreadsheet, as data in an XML document, as data in a database etc.

As an example, method 900 may include outputting an indication of an estimated emotional response relative to a distribution of emotional responses for typical, atypical but not autistic, and/or autistic subjects according to age, sex, and/or IQ. As another example, method 900 may include outputting an indication of detected gestures relative to a distribution of expected gestures for typical, atypical but not autistic, and/or autistic subjects according to age, sex, and/or IQ. As yet another example, method 900 may include outputting an indication of detected physiological responses relative to a distribution of physiological responses for typical, atypical but not autistic, and/or autistic subjects according to age, sex, and/or IQ. As still another example, method 900 may include, for each targeted autism construct, outputting an average amount of gaze time associated with the autism construct relative to a distribution of emotional responses for typical, atypical but not autistic, and/or autistic subjects according to age, sex, and/or IQ. Other examples of indications which may be output include the following: indications of calculated eye gaze times; indications of a quantified difference between a calculated eye gaze time and an expected eye gaze time associated with specific scenes and an autism construct; indications of a quantified difference between an estimated emotional salience of the subject and an expected emotional salience associated with specific scenes and an autism construct; indications of a difference between detected gestures of the subject and expected gestures associated with specific scenes and an autism construct; and indications of a difference between detected physiological responses of the subject and expected physiological responses associated with specific scenes and an autism construct.

At 944, method 900 may include determining if the subject's responses and/or inputs are substantially different from expected responses/inputs. If the subject's responses and/or inputs are substantially different from expected responses/inputs at 944, method 900 may proceed to 946 to output an indication of an autism condition. However, if the subject's responses and/or inputs are not substantially different from expected responses/inputs at 944, then method 900 may proceed to 948 to output an indication of an absence of an autism condition.

For example, an indication of an autism condition may be output in response to the gaze times associated with one or more autism constructs being less than predetermined thresholds for the autism constructs. For example, an indication of an autism condition may be output in response to a difference between a calculated eye gaze time and an expected eye gaze time associated with specific scenes and an autism construct greater than a predetermined threshold. As another example, a subject's response mapping across a plurality of autism constructs may be generated and correlated with a multi-factor autism assessment map (e.g., as shown in FIG. 8 described above) to calculate a score for the autism assessment test which may be used to indicate a presence or absence of autism conditions in the subject.

In some embodiments, the above described methods and processes may be tied to a computing system, including one or more computers. In particular, the methods and processes described herein, e.g., method 900 described above, may be implemented as a computer application, computer service, computer API, computer library, and/or other computer program product.

FIG. 10 schematically shows a non-limiting computing device 1000 that may perform one or more of the above described methods and processes. For example, FIG. 10 may represent one or more computing devices or processors included in system 100 shown in FIG. 1 described above. Computing device 1000 is shown in simplified form. It is to be understood that virtually any computer architecture may be used without departing from the scope of this disclosure. In different embodiments, computing device 1000 may take the form of a microcomputer, an integrated computer circuit, microchip, a mainframe computer, server computer, desktop computer, laptop computer, tablet computer, home entertainment computer, network computing device, mobile computing device, mobile communication device, gaming device, etc.

Computing device 1000 includes a logic subsystem 1002 and a data-holding subsystem 1004. Computing device 1000 may also include a display subsystem 1006, an audio subsystem 1008, one or more capture devices 1010, a communication subsystem 1012, and/or other components not shown in FIG. 10. Computing device 1000 may also optionally include user input devices such as manually actuated buttons, switches, keyboards, mice, game controllers, cameras, microphones, and/or touch screens, for example.

Logic subsystem 1002 may include one or more physical devices configured to execute one or more machine-readable instructions. For example, the logic subsystem may be configured to execute one or more instructions that are part of one or more applications, services, programs, routines, libraries, objects, components, data structures, or other logical constructs. Such instructions may be implemented to perform a task, implement a data type, transform the state of one or more devices, or otherwise arrive at a desired result.

The logic subsystem may include one or more processors that are configured to execute software instructions. For example, the one or more processors may comprise physical circuitry programmed to perform various acts described herein. Additionally or alternatively, the logic subsystem may include one or more hardware or firmware logic machines configured to execute hardware or firmware instructions. Processors of the logic subsystem may be single core or multicore, and the programs executed thereon may be configured for parallel or distributed processing. The logic subsystem may optionally include individual components that are distributed throughout two or more devices, which may be remotely located and/or configured for coordinated processing. One or more aspects of the logic subsystem may be virtualized and executed by remotely accessible networked computing devices configured in a cloud computing configuration.

Data-holding subsystem 1004 may include one or more physical, non-transitory, devices configured to hold data and/or instructions executable by the logic subsystem to implement the herein described methods and processes. When such methods and processes are implemented, the state of data-holding subsystem 1004 may be transformed (e.g., to hold different data).

Data-holding subsystem 1004 may include removable media and/or built-in devices. Data-holding subsystem 1004 may include optical memory devices (e.g., CD, DVD, HD-DVD, Blu-Ray Disc, etc.), semiconductor memory devices (e.g., RAM, EPROM, EEPROM, etc.) and/or magnetic memory devices (e.g., hard disk drive, floppy disk drive, tape drive, MRAM, etc.), among others. Data-holding subsystem 1004 may include devices with one or more of the following characteristics: volatile, nonvolatile, dynamic, static, read/write, read-only, random access, sequential access, location addressable, file addressable, and content addressable. In some embodiments, logic subsystem 1002 and data-holding subsystem 1004 may be integrated into one or more common devices, such as an application specific integrated circuit or a system on a chip.

FIG. 10 also shows an aspect of the data-holding subsystem in the form of removable computer-readable storage media 1016, which may be used to store and/or transfer data and/or instructions executable to implement the herein described methods and processes. Removable computer-readable storage media 1016 may take the form of CDs, DVDs, HD-DVDs, Blu-Ray Discs, EEPROMs, flash memory cards, and/or floppy disks, among others.

When included, display subsystem 1006 may be used to present a visual representation of data held by data-holding subsystem 1004. As the herein described methods and processes change the data held by the data-holding subsystem, and thus transform the the state of the data-holding subsystem, the state of display subsystem 1006 may likewise be transformed to visually represent changes in the underlying data. Display subsystem 1006 may include one or more display devices or surfaces utilizing virtually any type of technology. Such display devices or surfaces may be combined with logic subsystem 1002 and/or data-holding subsystem 1004 in a shared enclosure, or such display devices or surfaces may be peripheral display devices or surfaces.

When included, audio subsystem 1008 may be used to present an audio representation of data held by data-holding subsystem 1004. As the herein described methods and processes change the data held by the data-holding subsystem, and thus transform the state of the data-holding subsystem, the state of audio subsystem 1008 may likewise be transformed to represent changes in the underlying data via sounds or vibrations. Audio subsystem 1008 may include one or more devices or components capable of vibration, e.g., speakers or the like. Such devices may be combined with logic subsystem 1002 and/or data-holding subsystem 1004 in a shared enclosure, or such devices may be peripheral devices. In some embodiments, computing device 1000 may additionally include a haptic subsystem including one or vibration components which may be used to present haptic representations of data held by data-holding subsystem 1004.

Computing system 1000 may further include one or more capture devices 1010 configured to obtain various measurements. Examples of capture devices which may be included in computing system 1000 include one or more image capture devices (e.g., cameras or optical sensors), one or more audio capture devices (e.g., microphones), and various other sensors (examples of which are described above with regard to FIG. 1). In some examples, computing system 1000 may include a depth camera configured to capture video with depth information via any suitable technique (e.g., time-of-flight, structured light, stereo image, etc.).

When included, communication subsystem 1012 may be configured to communicatively couple computing device 1000 with one or more other computing devices and/or with various external sensors, components, or systems. Communication subsystem 1012 may include wired and/or wireless communication devices compatible with one or more different communication protocols. As non-limiting examples, the communication subsystem may be configured for communication via a wireless telephone network, a wireless local area network, a wired local area network, a wireless wide area network, a wired wide area network, etc. In some embodiments, the communication subsystem may allow computing system 1000 to send and/or receive messages to and/or from other devices via a network such as the Internet.

Computing system 1000 may further include various subsystems configured to execute one or more instructions that are part of one or more programs, routines, objects, components, data structures, or other logical constructs. Such subsystems may be operatively connected to logic subsystem 1002 and/or data-holding subsystem 1004. In some examples, such subsystems may be implemented as software stored on a removable or non-removable computer-readable storage medium.

It is to be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated may be performed in the sequence illustrated, in other sequences, in parallel, or in some cases omitted. Likewise, the order of the above-described processes may be changed.

The subject matter of the present disclosure includes all novel and nonobvious combinations and subcombinations of the various processes, systems and configurations, and other features, functions, acts, and/or properties disclosed herein, as well as any and all equivalents thereof

1. A computer-implemented method for performing an autism assessment of a subject, comprising:
presenting a movie animation on a display;
tracking the subject's eye gaze location on the display during specific scenes occurring within the animation, the specific scenes associated with an autism construct;
calculating an amount of time the subject's eye gaze tracks to predetermined regions on the display within the specific scenes to obtain a calculated eye gaze time associated with the specific scenes and the autism construct; and
outputting an indication of the calculated eye gaze time.

2. The method of claim 1, further comprising:
quantifying a difference between the calculated eye gaze time and an expected eye gaze time associated with the specific scenes and the autism construct; and
outputting an indication of the difference.

3. The method of claim 2, further comprising outputting an indication of an autism condition in response to the difference greater than a threshold.

4-9. (canceled)

10. The method of claim 1, further comprising:
detecting facial expressions of the subject during the specific scenes occurring within the animation;
estimating an emotional salience of the subject from the detected facial expressions;
quantifying a difference between the estimated emotional salience of the subject and an expected emotional salience associated with the specific scenes and the autism construct; and
outputting an indication of the difference between the estimated emotional salience of the subject and the expected emotional salience associated with the specific scenes and the autism construct.

11-16. (canceled)

17. The method of claim 1, further comprising:
detecting gestures of the subject during the specific scenes occurring within the animation;
quantifying a difference between the detecting gestures of the subject and expected gestures associated with the specific scenes and the autism construct; and
outputting an indication of the difference between the detecting gestures of the subject and expected gestures associated with the specific scenes and the autism construct.

18. The method of claim 1, further comprising:
detecting physiological responses of the subject during the specific scenes occurring within the animation;
quantifying a difference between the detected physiological responses of the subject and expected physiological responses associated with the specific scenes and the autism construct; and
outputting an indication of the difference between the detected physiological responses of the subject and expected physiological responses associated with the specific scenes and the autism construct.

19. (canceled)

20. The method of claim 1, wherein the autism construct comprises a joint attention autism construct and presenting a movie animation on the display comprises presenting animated content on the display to initiate a predetermined expected redirection of the subject's eye gaze to the predetermined regions on the display within the specific scenes.

21. (canceled)

22. The method of claim 1, wherein the autism construct comprises an emotion recognition autism construct and presenting a movie animation on the display comprises displaying at least a first character and a second character, the first character exhibiting a first emotion and the second character exhibiting a second emotion, and wherein the method further comprises initiating a predetermined expected redirection of the subject's eye gaze to a character exhibiting the first emotion and wherein the predetermined regions correspond to a location of the first character on the display.

23. The method of claim 1, wherein the autism construct comprises an emotion recognition construct and presenting a movie animation on a display comprises displaying a primary character exhibiting an emotion in reaction to a scenario and wherein the predetermined regions correspond to a location of the primary character on the display.

24. The method of claim 1, wherein the autism construct comprises a theory of mind autism construct, and presenting a movie animation on the display comprises displaying an animated character viewing an object being hidden at a first location then displaying a movement of the hidden object out of view of the character to a second hidden location and the method further comprises initiating a predetermined expected redirection of the subject's eye gaze to the hidden object and wherein the predetermined regions correspond to the second hidden location of the object.

25. The method of claim 1, wherein the autism construct comprises a social engagement autism construct and presenting a movie animation on the display comprises displaying at least a first animated character and a second animated character or object, the first character exhibiting an active behavior and the second character or object exhibiting a passive behavior, and wherein the predetermined regions include regions on the display corresponding to a location of the first character on the display.

26. The method of claim 25, wherein presenting a movie animation on the display further comprises displaying the first character exhibiting a passive behavior and the second character or object exhibiting an active behavior, and wherein the predetermined regions include regions on the display corresponding to a location of the second character or object on the display.

27-28. (canceled)

29. The method of claim 1, wherein the autism construct comprises a narrative autism construct, and presenting a movie animation on the display comprises displaying a sequence of events on the display and the method further comprises: following display of the sequence of events, prompting the subject for input describing what happened during the sequence of events.

30. The method of claim 1, wherein the autism construct comprises a creativity and imagination autism construct, and presenting a movie animation on the display comprises displaying a sequence of events on the display and the method further comprises: following display of the sequence of events, prompting the subject for input describing events subsequent to the displayed sequence of events.

31. The method of claim 1, wherein the autism construct comprises an imitation autism construct and presenting a movie animation on the display comprises displaying a character performing an action and the method further comprises: prompting the subject to imitate the action performed by the character, detecting the subject's action following the prompt, and comparing the detected subject's action to the action performed by the character.

32. A computer-implemented method for performing an autism assessment of a subject, comprising:
displaying an animation on a display, the animation comprising a plurality of scenes associated with a plurality of autism constructs;
detecting the subject's eye gaze location on the display during scenes in the plurality of scenes, where each scene in the plurality of scenes is associated with an autism construct in the plurality of autism constructs;
for each scene in the plurality of scenes, calculating an amount of time the subject's eye gaze location on the display is within predetermined regions on the display within the scene and associating the calculated amount of time with the autism construct associated with the scene; and
for each autism construct in the plurality of autism constructs, calculating an average amount of time associated with the autism construct from the amounts of time associated with the autism construct and outputting an indication of the average amount of time associated with the autism construct.

33. The method of claim 32, further comprising:
detecting facial expressions of the subject during display of a scene in the plurality of scenes;
estimating an emotional response of the subject from the detected facial expressions;
quantifying a difference between the estimated emotional response of the subject and an expected emotional response associated with scene and the autism construct associated with the scene; and
outputting an indication of the difference between the estimated emotional response of the subject and the expected emotional response associated with scene and the autism construct associated with the scene.

34. The method of claim 32, further comprising:
detecting gestures of the subject during display of a scene in the plurality of scenes;
quantifying a difference between the detecting gestures of the subject and expected gestures associated with scene and the autism construct associated with the scene;
outputting an indication of the difference between the detecting gestures of the subject and the expected gestures associated with scene and the autism construct associated with the scene.

35. The method of claim 32, further comprising:
detecting physiological responses of the subject during display of a scene in the plurality of scenes;
quantifying a difference between the detected physiological responses of the subject and expected physiological responses associated with the scene and the autism construct associated with the scene;
outputting an indication of the difference between the detected physiological responses of the subject and the expected physiological responses associated with the scene and the autism construct associated with the scene.

36. The method of claim 32, wherein the plurality of autism constructs include one or more of a joint attention autism construct, an emotion recognition autism construct, a shared affect autism construct, a theory of mind autism construct, a social engagement autism construct, a narrative autism construct, a creativity and imagination autism construct, and an imitation autism construct.

37-38. (canceled)
